(19) United States
(12) Patent Application Publication
(10) Pub. No.: US 2025/0099763 A1
(43) Pub. Date: Mar. 27, 2025

(54) Methods, Systems, Devices and Components for the Treatment of Stroke in a Patient with Interferential Cranial Electrical Stimulation

(71) Applicant: Makous Research, LLC, Carsbad, CA (US)

(72) Inventor: James Carl Makous, Carlsbad, CA (US)

(21) Appl. No.: 18/657,444

(22) Filed: May 7, 2024

Related U.S. Application Data
(60) Provisional application No. 63/540,087, filed on Sep. 24, 2023.

(51) Int. Cl. A61N 1/36; A61N 1/04; A61N 1/05; A61N 1/32

(52) U.S. Cl. CPC A61N 1/3606; A61N 1/0456; A61N 1/0504; A61N 1/323; A61N 1/36157; A61N 1/36171; A61N 1/36175

(57) ABSTRACT

Described and disclosed herein are various embodiments of methods, systems, devices and components that are configured to treat stroke in patients who have experienced strokes using interferential electrical stimulation techniques.

### RELATED APPLICATIONS, BENEFITS AND CLAIMS TO PRIORITY

This application is related to, and claims priority and other benefits from, U.S. Provisional Patent Application Ser. No. 63/540,087 entitled “Methods, Systems, Devices and Components for the Treatment of Stroke in a Patient with Interferential Cranial Electrical Stimulation” to Makous filed on Sep. 24, 2023 (hereafter “the '087 provisional patent application”), and claims priority and other benefits therefrom. The '087 provisional patent application is hereby incorporated by reference herein, in its entirety, to provide continuity of disclosure.

### FIELD OF THE INVENTION

The present invention relates to systems, devices, components and methods for treating a patient who has experienced at least one stroke with interferential cranial electrical stimulation techniques.

### BACKGROUND

Disclosed are methods, systems, devices and components for the treatment of stroke with interferential cranial electrical stimulation. Every year, more than 795,000 people in the United States have a stroke, and every 3 minutes and 14 seconds someone in the U.S. dies of a stroke, Stroke-related costs in the United States came to nearly $56.5 billion between 2018 and 2019, which includes the cost of health care services, medicines to treat stroke, and missed days of work. Stroke is a leading cause of serious long-term disability, including hemiparesis, which is a serious motor impairment, affecting 65% of stroke victims.

What is needed are improved methods, systems, devices and components for treating patients who have experienced a stroke.

### SUMMARY

According to some embodiments, there is provided a method of electrically stimulating a portion of a patient's brain to treat stroke, the method comprising positioning a plurality of electrodes on a patient's skull; delivering first interferential electrical stimulation signals that combine to form at least one beat frequency associated therewith through at least a first pair or more of the plurality of electrodes to a region of the patient's brain that has been identified as having been affected or likely to have been affected by a stroke; at least one of monitoring, measuring, sensing and recording at least one of verbal responses, motor movement responses, and muscle twitch responses of the patient in response to delivery of the first interferential electrical stimulation signals through the at least first pair or more of the plurality of electrodes to the region of the patient's brain that has been identified as having been affected or likely to have been affected by the stroke; selecting, from among the plurality of electrodes, a second pair or more of electrodes different from the first pair or more of electrodes through which to deliver second interferential electrical stimulation signals to the region of the patient's brain that has been identified as having been affected or likely to have been affected by the stroke; at least one of monitoring, sensing, measuring, and recording at least one of verbal responses, motor movement responses, and muscle twitch responses of the patient in response to delivery of the second interferential electrical stimulation signals through the at least second pair or more of the plurality of electrodes to the region of the patient's brain that has been identified as having been affected or likely to have been affected by the stroke; subsequently and sequentially repeating delivery of interferential stimulation signals to the region of the patient's brain that has been identified as having been affected or likely to have been affected by the stroke using different pairs or more of electrodes, each pair or more of electrodes being different from a preceding pair or more of electrodes; for each pair or more of electrodes through which interferential electrical stimulation signals are delivered to the region of the patient's brain that has been identified as having been affected or likely to have been affected by the stroke, at least one of monitoring, sensing, measuring and recording at least one of verbal responses, motor movement responses, and muscle twitch responses of the patient; determining, from among the monitored or measured limb movements of the patient corresponding to interferential electrical stimulation signals delivered through the different pairs or more of electrodes, one or more pairs of electrodes that provide optimum verbal responses, motor movement responses, and muscle twitch responses of the patient, and selecting the one or more pairs of electrodes that provide at least one of optimum verbal responses, motor movement responses, and muscle twitch responses of the patient for chronic or episodic interferential electrical stimulation of the region of the patient's brain that has been identified as having been affected or likely to have been affected by the stroke, thereby to treat stroke in the patient.

Such a method may further comprises one or more of: (a) wherein the interferential electrical stimulation signals are delivered simultaneously through the at least first pair or more of the plurality of electrodes; (b) wherein at least two different interferential electrical stimulation signals are delivered through at least two pairs or more of the plurality of electrodes; (c) further comprising providing the interferential electrical stimulation signals with a carrier frequency ranging between about 1 kHz and about 50 KHz; (d) further comprising providing the interferential electrical stimulation signals with a beat frequency ranging between about 1 Hz and about 300 Hz; (e) further comprising providing the interferential electrical stimulation signals with an amplitude ranging between about 0.5 mA and about 50 mA; (f) further comprising providing the interferential electrical stimulation signals with an on duty cycle selected from the group consisting of about 1 second, about 2 seconds, about 5 seconds, about 10 seconds, about 20 seconds, about 30 seconds, and about 1 minutes; (g) further comprising providing the interferential electrical stimulation signals with an off duty cycle selected from the group consisting of about 1 second, about 2 seconds, about 5 seconds, about 10 seconds, about 20 seconds, about 30 seconds, and about 1 minute; (h) wherein the region of the patient's brain that has been identified as having been affected or likely to have been affected by the stroke is at or near the motor strip of the cortex of the patient's brain (Brodman's area 4); (i) wherein the region of the patient's brain that has been identified as having been affected or likely to have been affected by the stroke is between about 0.5 centimeters and about 10 centimeters beneath the patient's skull; (j) wherein the plurality of electrodes are positioned subcutaneously between the patient's skin and skull; (k) wherein the plurality of electrodes are positioned transdermally atop the patient's skin and skull; (l) wherein the plurality of electrodes are positioned epidurally or subdurally as regards the patient's skull and brain; (m) wherein the plurality of electrodes comprise between 2 electrodes and 64 electrodes; (n) wherein the plurality of electrodes are mounted on a substrate or patch configured for placement on or in the patient's skull; (o) wherein the steps of delivering, monitoring, selecting, and repeating are controlled and executed by a stimulation algorithm and method; (p) wherein optimum verbal response of the patient and optimum movements in the patient's limb or limbs are determined on the basis of at least one of a range of motion of the limb or limbs, a type of muscular contraction occurring in the limb or limbs, a direction of motion of the limb or limbs, and a degree, type or volume of the patient's verbal response to electrical stimulation; (q) further comprising delivering chronic or episodic interferential electrical stimulation signals through the one or more pairs of electrodes that have been determined to provide optimum or largest limb movements in the patient, thereby to treat stroke in the patient, for a period of time ranging between about 1 day and about 6 months, and (r) further comprising administering physical therapy to the patient.

In another embodiment, there is provided a system for electrically stimulating a portion of a patient's brain to treat stroke, the system comprising a plurality of electrodes, a pulse generator, at least one lead configured to operably connect the pulse generator to the plurality of electrodes, and a sensor configured to at least one of monitor, measure, sense and record at least one of verbal responses, motor movement responses, and muscle twitch responses of the patient in response to delivery of first interferential electrical stimulation signals through at least a first pair or more of the plurality of electrodes, the plurality of electrodes being configured to be placed on a patient's skull, the pulse generator being configured to deliver the first interferential electrical stimulation signals through the at least one lead to at least the first pair or more of the plurality of electrodes to a region of the patient's brain that has been identified as having been affected or likely to have been affected by a stroke, the sensor means for monitoring, measuring, sensing or recording the at least one of verbal responses, motor movement responses, and muscle twitch responses of the patient in response to delivery of first interferential electrical stimulation signals through at least a first pair or more of the plurality of electrodes to the region of the patient's brain that has been identified as having been affected or likely to have been affected by the stroke being operably connected to the system, the system being configured to select from among the plurality of electrodes a second pair or more of electrodes different from the first pair or more of electrodes through which to deliver second interferential electrical stimulation signals to the region of the patient's brain that has been identified as having been affected or likely to have been affected by the stroke, the system and the sensor further being configured to at least one of monitor, sense, measure and record at least one of verbal responses, motor movement responses, and muscle twitch responses of the patient in response to delivery of the second interferential electrical stimulation signals through the at least second pair or more of the plurality of electrodes to the region of the patient's brain that has been identified as having been affected or likely to have been affected by the stroke, the system still further being configured to subsequently and sequentially repeat delivery of interferential stimulation signals to the region of the patient's brain that has been identified as having been affected or likely to have been affected by the stroke using different pairs or more of electrodes, each pair or more of electrodes being different from a preceding pair or more of electrodes, thereby to treat stroke in the patient.

Such a system may further comprise one or more of: (a) wherein one of a user selects the second pair or more of electrodes and instructions loaded into a non-transient memory of the pulse generator are employed to select the second pair or more of electrodes; (b) wherein the system is configured such that for each pair or more of electrodes through which interferential electrical stimulation signals are delivered to the region of the patient's brain that has been identified as having been affected or likely to have been affected by the stroke, at least one of monitoring, measuring, and sensing at least one of verbal responses, motor movement responses, and muscle twitch responses of the patient is carried out while the interferential electrical stimulation signals are being delivered to the region of the patient's brain; (c) wherein the system is configured such that from among the monitored, sensed, measured and recorded verbal responses, motor movement responses, and muscle twitch responses of the patient, one or more pairs of electrodes that provide optimum or largest verbal responses of and limb movements in the patient are selected for chronic or episodic interferential electrical stimulation of the region of the patient's brain that has been identified as having been affected or likely to have been affected by the stroke, thereby to treat stroke in the patient; (d) wherein the interferential electrical stimulation signals are delivered simultaneously through the at least first pair or more of the plurality of electrodes; (e) wherein at least two different interferential electrical stimulation signals are delivered through at least two pairs or more of the plurality of electrodes; (f) further comprising providing the interferential electrical stimulation signals with a carrier frequency ranging between about 1 kHz and about 50 KHz; (g) further comprising providing the interferential electrical stimulation signals with a beat frequency ranging between about 1 Hz and about 300 Hz; (h) further comprising providing the interferential electrical stimulation signals with an amplitude ranging between about 0.5 mA and about 50 mA: (i) further comprising providing the interferential electrical stimulation signals with an on duty cycle selected from the group consisting of about 1 second, about 2 seconds, about 5 seconds, about 10 seconds, about 20 seconds, about 30 seconds, and about 1 minutes; (j) further comprising providing the interferential electrical stimulation signals with an off duty cycle selected from the group consisting of about 1 second, about 2 seconds, about 5 seconds, about 10 seconds, about 20 seconds, about 30 seconds, and about 1 minute; (k) wherein the region of the patient's brain that has been identified as having been affected or likely to have been affected by the stroke is at or near the motor strip of the cortex of the patient's brain (Brodman's area 4); (l) wherein the region of the patient's brain that has been identified as having been affected or likely to have been affected by the stroke is between about 0.5 centimeters and about 10 centimeters beneath the patient's skull; (m) wherein the plurality of electrodes are positioned subcutaneously between the patient's skin and skull; (n) wherein the plurality of electrodes are positioned transdermally atop the patient's skin and skull; (o) wherein the plurality of electrodes are positioned epidurally or subdurally as regards the patient's skull and brain; (p) wherein the plurality of electrodes comprise between 2 electrodes and 64 electrodes; (q) wherein the plurality of electrodes are mounted on a substrate or patch configured for placement on or in the patient's skull; (r) wherein at least one of the steps of delivering, monitoring, sensing, measuring, recording, selecting, and repeating is controlled and executed by a stimulation algorithm and method; (s) wherein optimum verbal responses, motor movement responses, and muscle twitch responses of the patient are determined on the basis of at least one of a range of motion of the limb or limbs, a type of muscular contraction occurring in the limb or limbs, a direction of motion of the limb or limbs, and a degree, type or volume of the patient's verbal response to electrical stimulation; (t) further comprising delivering chronic or episodic interferential electrical stimulation signals through the one or more pairs of electrodes that have been determined to provide at least one of optimum verbal responses, motor movement responses, and muscle twitch responses of the patient, thereby to treat stroke in the patient, for a period of time ranging between about 1 day and about 6 months, and (u) further comprising administering physical therapy to the patient.

Other embodiments, permutations, combinations and variations of the foregoing are also contemplated.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

Different aspects of the various embodiments will become apparent from the following specification, drawings and claims in which:

FIG. 1 shows a block diagram of one embodiment of an interferential electrical stimulation system 10 configured to deliver therapy to a patient who has suffered from a stroke;

FIG. 2 shows a block diagram of another embodiment of an interferential electrical stimulation system 10 configured to deliver therapy to a patient who has suffered from a stroke;

FIG. 3 shows a schematic view of one embodiment of providing interferential electrical stimulation to a portion 50 of a patient's brain that has been affected by a stroke;

FIG. 4 shows another schematic view of one embodiment of providing interferential electrical stimulation to a portion 50 of a patient's brain 52 that has been affected by a stroke;

FIG. 5 shows one embodiment of a lead and electrode configuration 40/42;

FIG. 6 shows the embodiment of lead and electrode configuration 40/42 of FIG. 5 in a test setup using animal tissue;

FIG. 7 shows test results obtained using the lead and electrode configuration 40/42 of FIGS. 5 and 6;

FIG. 8 shows further test results obtained using the electrode configurations of FIGS. 5 and 6;

FIG. 9 illustrates various examples or different embodiments of surface electrode configurations 40/42;

FIG. 10 shows a superior view of the brain of a patient;

FIGS. 11 and 12 show the cerebral cortex of a patient with electrode arrays 40/42 overlain parallel and perpendicular to the motor cortex strip of patient's brain 52, respectively;

FIGS. 13 through 15 show some illustrative or representative electrode configurations 40/42 that may be employed in some embodiments of the interferential electrical stimulation systems, devices, components and methods described and disclosed herein;

FIG. 16 shows an example of skin impedance (Ohms) as a function of electrical stimulation sinusoidal frequency (Hz)

FIGS. 17-19 illustrate embodiments of method 100 and electrode configurations 40/42 for providing interferential electrical stimulation therapy to a patient who has suffered from a stroke.

The drawings are not necessarily to scale. Like numbers refer to like parts or steps throughout the drawings. Electrode polarity in all embodiments may vary depending on the patient's needs.

### DETAILED DESCRIPTIONS OF SOME EMBODIMENTS

Described and disclosed herein are various embodiments of methods, systems, devices and components associated with treating stroke in a patient through the targeted means of interferential cranial electrical stimulation. These various embodiments include temporary or permanent interferential cranial electrical stimulation techniques, as well as implantable and non-implantable interferential cranial electrical stimulation techniques. It is also contemplated that the various embodiments of methods, systems, devices and components relating to the treatment of stroke and interferential electrical stimulation can be employed in different combinations, permutations and variations.

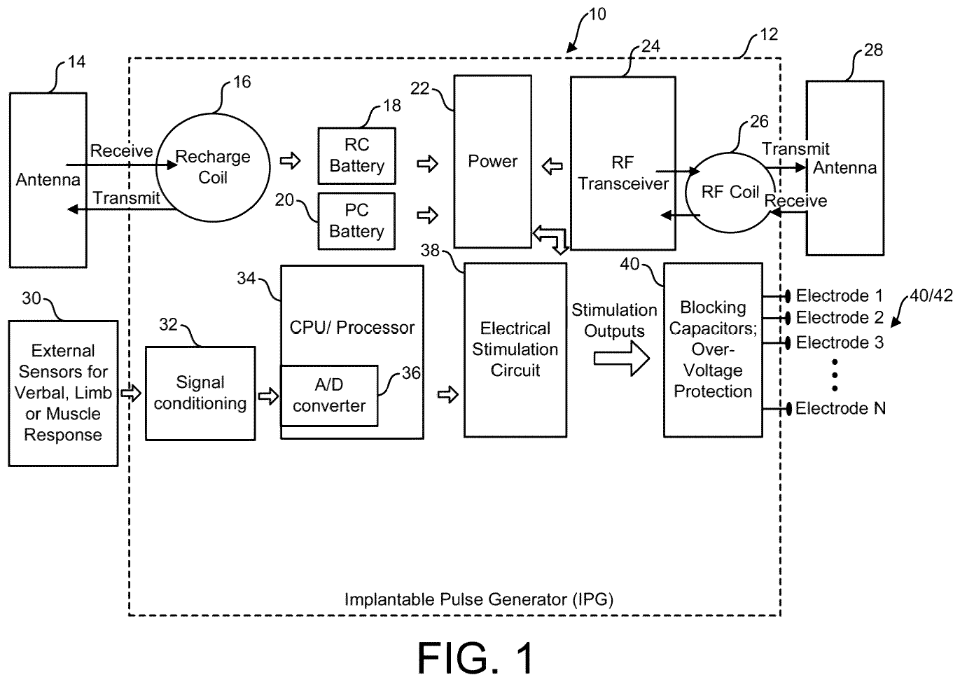

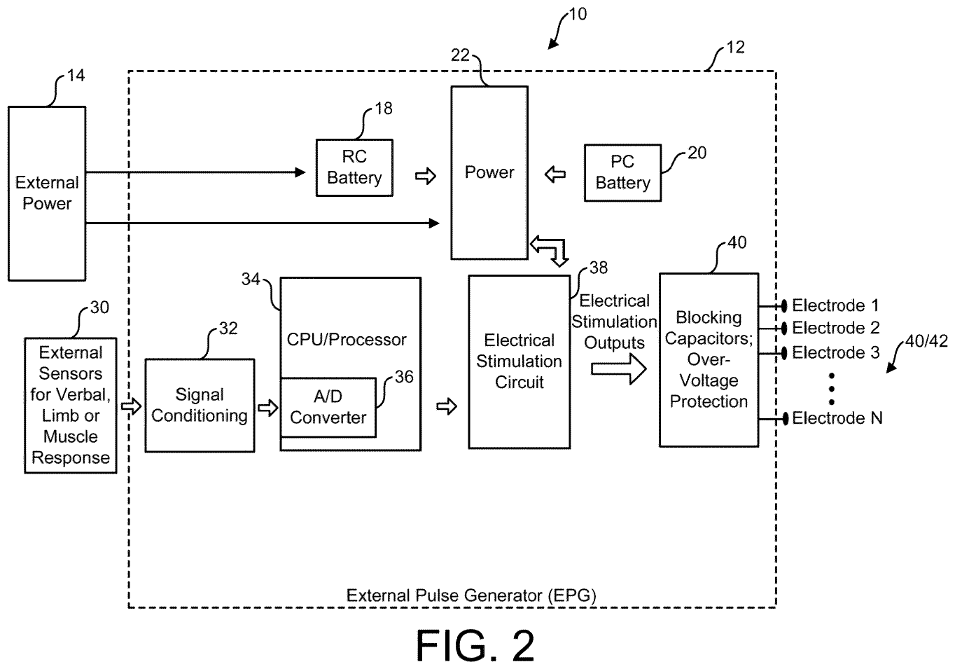

Referring now to FIGS. 1 and 2, there are shown block diagrams of electrical circuitry that may be employed in implantable pulse generator (IPG) and external pulse generator (EPG) embodiments, respectively. FIG. 1 shows one embodiment of an IPG that may be employed to deliver interferential electrical stimulation signals described in more detail below, whereas FIG. 2 shows one embodiment of an EPG that may be employed to deliver interferential electrical stimulation signals (also described in more detail below).

In two embodiments, and with reference to FIGS. 1 and 2, there are shown an implantable pulse generator (IPG) embodiment in FIG. 1 and an external pulse generator (EPG) embodiment in FIG. 2 of neurostimulator system 10 configured to electrically stimulate one or more portions of a patient's brain that has been affected by a stroke. IPG 12 and EPG 12 may be configured to deliver different types of stimulation signals to or near a patient's brain, more about which is said below, including burst signals, continuous signals, discrete signals, and signals having different types of waveforms such as ramped signals, square wave signals, sinusoidal signals, triangular wave signals, biphasic signals, and so on. IPG and EPG 12 each comprise a housing, at least one medical electrical lead 40 comprising multiple stimulation electrodes 42, pulse generation or stimulation circuitry 38 operably connected to lead 40 and electrodes 42, power, energy and/or electrical charge storage, charging/recharging, and/or delivery circuitry 16/18/20/22 operably connected to pulse generation or stimulation circuitry 38 and other portions of circuitry included in IPG/EPG 12 such as CPU/Processor 34. In some embodiments, power, energy and/or electrical charge storage, charging/recharging, and/or delivery circuitry 16/18/20/22 is configured to receive power, energy or electrical charge signals transcutaneously from an external power source and external power transmitting circuitry associated therewith. In some embodiments, power, energy and/or electrical charge storage, charging/recharging, and/or delivery circuitry 16/18/20/22 may comprise a primary or secondary (rechargeable) battery or capacitor to provide electrical power to circuitry.

In some embodiments, the various components disposed within IPG/EPG 12 are typically disposed within a housing, which may or may not be hermetically sealed. Hermetic sealing of the housing, if desired, may be accomplished in a number of ways, such as by disposing a hermetic coating or layer over the interior or exterior surfaces of the housing, or forming the housing out of a suitable malleable, bendable, or shapeable metal or metal alloy. The components and circuitry disposed inside housing may also be sealed and potted therein using epoxy, silicone, a polymer, or other suitable materials. IPG/EPG 12 may be sized, shaped and configured to be implanted on or in the skull, head, neck, leg, torso, back, trunk or shoulder of the patient atop or beneath the patient's skin, and lead and electrodes 40/42 may be sized, shaped and configured to be implanted beneath the patient's skin or skull, or epidurally or subdurally, and positioned adjacent to, in contact with, or in operative positional relationship to, one or more target portions of the patient's brain.

Continuing to refer to FIGS. 1 and 2, there are shown block diagrams of two embodiments of a cranial electrical interferential signal stimulation system 10, which as shown comprise IPG 12 or EPG 12 including one or more medical electrical leads 40, and which according to some embodiments may also comprise a clinician programmer or CP (not shown in FIG. 1 or 2), a patient programmer or PP (not shown in FIG. 1 or 2), and a central server, remote computer, and/or local computer (also not shown in FIG. 1 or 2). Other components of system 10 are also contemplated, more about which is said below. IPG 12 or EPG 12 may include one or more leads 40 to which the circuitry internal to IPG 12 or EPG 12 is operably connected. One or more such leads 40 may be operably connected to IPG 12 or EPG 12. Thus, IPG/EPG 12 contains or may be operably connected to the proximal ends of one or more medical electrical leads 40, which according to one embodiment are implantable leads configured for chronic, sub-chronic or temporary placement beneath a patient's skin, beneath the skull of the patient in the patient's brain, or atop the patient's skin so as to electrically stimulate a portion of the patient's brain that has been determined to have been subjected to a stroke. In some embodiments, IPG/EPG 12 features operational and/or stimulation parameters that can be programmed, and in other embodiments such parameters are predetermined, or are predetermined but selectable from instructions stored in a non-transient memory of stimulation control circuitry 34/36, as further described below.

In the embodiments shown in FIGS. 1 and 2, and in one embodiment, the distal end of lead 40/42 is configured to be situated near, above, below, or to a side of a portion of the brain that has been determined to have suffered a stroke and provides electrical stimulation signals originating from IPG 12 or EPG 12 to or near, by way of non-limiting example, such portion of the patient's brain. Other portions of the patient's brain may also be stimulated by system 10.

In some embodiments, the electrical stimulation parameters, therapy delivery, and/or operational parameters of IPG/EPG 12 may be programmed by a CP under the control of a physician or other health care provider and/or may be stored and preprogrammed in a non-transient memory of IPG/EPG 12 (included, for example, in CPU/Processor 34 and/or Stimulation Circuit or control circuitry 38—see FIGS. 1 and 2). Optional patient programmer PP may operate under the control of the patient, and may also be configured to permit a patient or health care professional to turn IPG/EPG 12 on or off, to change electrical stimulation parameters (in some embodiments within certain limits), or to effect other changes in the operation of IPG/EPG 12. In one embodiment, a CP is configured to permit a physician or other health care provider to program a PP via wireless or other communication and connection means (e.g., Bluetooth, RF, telemetry, inductive or magnetic coupling, cable, etc.). A remote or local server or computer may also be configured to receive and/or transmit data, programming instructions, and the like from and to the CP and/or PP, as well as to process, analyze, and facilitate interpretation of such data.

In the various embodiments, bipolar electrodes (or a set of two electrodes) may be employed in lead 40, although other configurations of electrodes for lead(s) 40 are also contemplated, such as unipolar electrodes, tri-polar electrodes, using the IPG or EPG as a ground electrode or anode or cathode, and more.

In some embodiments, IPG/EPG 12 includes a conventional connector block to which the proximal ends of one or more lead(s) 40 and/or lead extensions are connected. In other embodiments, a single lead 40 forms a portion of IPG/EPG 12 and no connector block is required. In still other embodiments, multiple leads 40 form a portion of IPG/EPG 12 and no connector block may be required.

If provided as part of system 10, CP can be a tablet device configured to communicate wirelessly (e.g., via Bluetooth) with IPG/EPG 12 and/or the patient's PP (which can be a smart phone). In some embodiments, a PP is configured to permit a patient to activate, deactivate, program and/or adjust the electrical stimulation parameters and operation of IPG/EPG 12.

Continuing to refer to FIG. 1, there is shown a block diagram of one embodiment of IPG 12, where power supply/battery or capacitor recharge antenna 14 is operably connected to recharge coil 16, which in turn is operably connected to recharge RC (rechargeable) battery 18 and/or battery PC (primary cell) 20. In one embodiment, power circuitry 22 is configured to handle the distribution of electrical power to the various circuitry components disposed in IPG 12. RF transceiver 24 is operably connected to RF (radio frequency) coil 26 and communication antenna 28 and is configured to receive and/or transmit information, instructions and/or data wirelessly from or to external devices such as computers, servers, a CP or a PP. In one embodiment, external sensor(s) for verbal, limb or finger/hand response 30 are employed to measure, sense, monitor, and/or permit the recording at least one of verbal responses of the patient and motor movements or twitches of one or more limbs or muscles of the patient in response to delivery of first interferential electrical stimulation signals through at least a first pair or more of the plurality of electrodes. Motor movements of the patient can include the patient pressing a button or trigger on an external device to indicate a favorable or unfavorable response to interferential electrical stimulation signals delivered to the patient's brain, and may also include sensing a patient's limb movement. In one embodiment, once optimum or desired limb or muscle movement or twitch parameters have been determined for a given patient, the electrical stimulation amplitude(s) delivered to the patient can be set below motor threshold (MT), say at 50% or 80% of MT or at some other stimulation amplitude with respect to MT.

Continuing to refer to FIG. 1, an external power transmitting device may be configured to provide power, energy or charge transcutaneously or otherwise IPG 12 through antenna 14. In some embodiments, a PP may include the functionality of an external power transmitting device. In other embodiments, an external device may be configured to transfer power, energy or charge to IPG 12 so that it may operate and electrically stimulate a predetermined portion of the patient's brain that has been determined to have suffered a stroke, or so that it may charge a capacitor or battery in IPG 12, or both. In still further embodiments, power receiving circuitry contained in power circuit 22 and/or batteries 18 and/or 20, or otherwise can comprise one or more of electrical charge storage circuitry, one or more internal induction coils configured to receive electrical power transcutaneously from one or more corresponding external induction coils, or one or more wireless, RF, acoustic, piezoelectric, thin film bulk wave acoustic resonators (FBAR), or microwave energy receiving circuits.

In some embodiments, ASICs or other integrated circuits may be employed to provide the functionalities and operations of one or more of circuitry 16, 22, 24, 26, 28, 32, 34, 38, and 40.

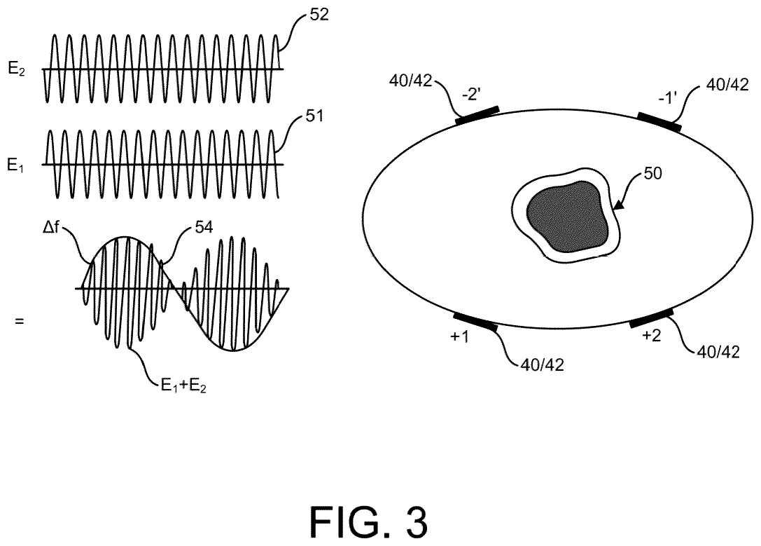

Referring now to FIG. 3, two frequencies of similar amplitude, but slightly disparate frequencies, can add and subtract to create a beat frequency, which when applied to the human body is referred to herein as interferential electrical stimulation. For example, and in one embodiment, a frequency of 1000 Hz on one channel (E₁, 51) and a frequency of 1100 Hz on another channel (E₂, 52) can create a volume of tissue activated (VTA) according to the 100 Hz difference frequency between the two signals (Δf, 54). A VTA created by the beat frequency is denoted by the red cloud (50) in FIG. 3, which is a portion of a patient's brain 50 that has been identified or determined to have been subjected to a stroke, or likely to have been subjected to a stroke. The VTA may extend beyond the portion of a patient's brain 50 that has been identified or determined to have been subjected to a stroke, or likely to have been subjected to a stroke. Note that the portion of a patient's brain 50 that has been identified or determined 30 to have been subjected to a stroke, or likely to have been subjected to a stroke, often corresponds to or includes some portion of Broadman's Area 4 (53), more about which is said below.

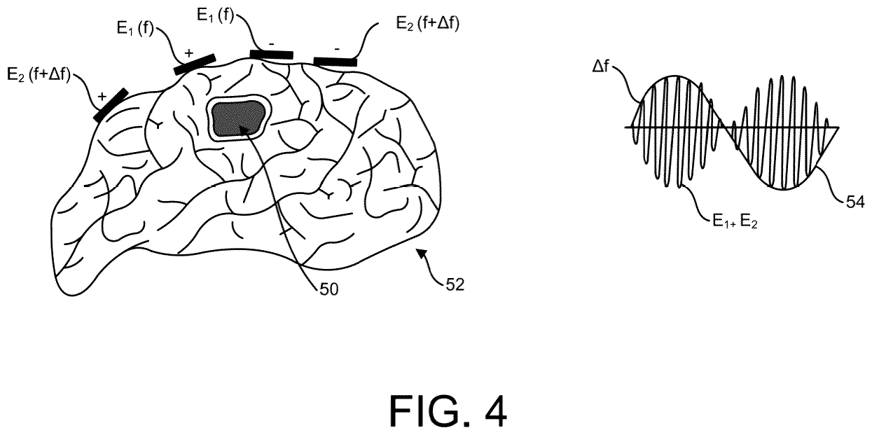

Referring now to FIG. 4 below, interferential electrical stimulation (IFS) is shown schematically applied to a portion 50 of a patient's brain. E₁(f) and E₂(f+Δf) represent the electric filed created by two interfering frequencies separated by an amount Δf. A hypothetical VTA is represented by the red cloud in FIG. 4. We describe and disclose here the stimulation of the cerebral cortex via IFS in some embodiments, which according to one embodiment is applied under the scalp but above the skull, to treat stroke. In some other embodiments, the stimulation of the cerebral cortex via IFS is applied epidurally, subdurally, on top of the scalp, or beneath the skull inside the patient's brain near brain portion 50. Electrode polarity may vary depending upon the patient needs.

Below are set forth further details and descriptions of various embodiments of the interferential cranial electrical stimulation methods, systems, devices and components described and disclosed herein, including FIGS. 4-27 described below.

Now described are some examples of general electrical stimulation parameters and techniques that can be applied to the interferential electrical stimulation methods, components, devices and systems described and disclosed herein. Note that in some embodiments the following electrical stimulation parameters may be employed: a carrier frequency between 1 kHz and 50 KHz; a beat frequency between 1 Hz and 500 Hz; a stimulation amperage delivered to electrodes 42 by IPG/EPG 12 ranging between about 0.5 mA and about 50 mA; a duty cycle having a cycle on time of about 1 second, about 2 seconds, about 5 seconds, about 10 seconds, and on continuously; a duty cycle off time of about 1 second, about 2 seconds, about 5 seconds, about 10 seconds; ramping electrical stimulation signals up and/or down; ramping stimulation signals up and down for periods of time ranging between about 0 seconds and about 10 seconds; electrically stimulating the patient's cortex or a portion of the patient's cortex; electrically stimulating the patient's cortex or a portion of the patient's cortex near or at the stroke location; electrically stimulating the patient's cortex or a portion of the patient's cortex by straddling or being parallel to the patient's motor strip (hand, leg, or other); electrically stimulating subcutaneously, between the patient's skin and skull, transdermally, epidurally, and/or subdurally.

Some examples of electrodes and electrode arrays that may be employed to deliver interferential electrical stimulation to a patient's brain include, but are not limited to, 2 or more stimulation electrodes, or 4, 6, 8, 10, 12, 14, 16, 18, 20, 22, 24, 26, 28, 30, 32, 34, or 36 or more stimulation electrodes in total; patch electrode arrays, multiple electrode patches depending upon stroke dimensions, electrode diameters ranging between about 1 mm and about 10 mm or between about 1 mm and about 50 mm, and square, circular, rectangular, triangular, oval and polygonal electrode shapes.

Some of the embodiments described and disclosed herein permit the treatment of stroke using interferential electrical stimulation techniques at depths ranging between about 0.5 cm and about 10 cm beneath the surface of a patient's skull. The region of the patient's brain that has suffered a stroke can be identified using different techniques, including X-rays, CAT scans, CT scans, PET scans, DTI scans and MRI scans. The region of the patient's brain that has suffered a stroke can be characterized using visual or other imaging techniques to determine stroke region length, width and depth, and/or motor strip width and lesion size or other characteristics, which in turn can be used to determine tailored or optimal electrode stimulation geometry, electrode configuration, and interferential electrical stimulation parameters. See, for example, Ono M., Kubik S., Abernathy C, “Atlas of the cerebral sulci,” New York, Georg Thieme Verlag; 1990.

Once initial stimulation electrode type and configuration and interferential electrical stimulation parameters have been settled on, different stimulation parameters and electrode configurations can be cycled through to optimize the delivery of interferential electrical stimulation therapy. Motor, twitching and/or verbal response of the patient via arm or leg motion, muscle or motor-induced motion anywhere in the patient's body, movement or twitching of the eye, head, neck, torso, or face or muscles associated therewith, and/or range or type of motion can be employed to optimize the delivery of interferential electrical stimulation therapy, and may be monitored, sensed, measured or otherwise observed using electromyographic (EMG) techniques and devices, one or more accelerometers, patient feedback (e.g., a hand-held clicker or button), physician feedback, and/or motion or sound sensors configured to detect such twitching, muscle, motor or other movement. A search algorithm may also be employed, more about which is said below, to optimize electrical stimulation parameters. In some embodiments, the search for optimum electrode configuration and interferential electrical stimulation parameters begins by employing the most likely or standard parameters. Random hunting techniques, sequential search techniques, and adaptive artificial intelligence (AI) techniques may also be employed.

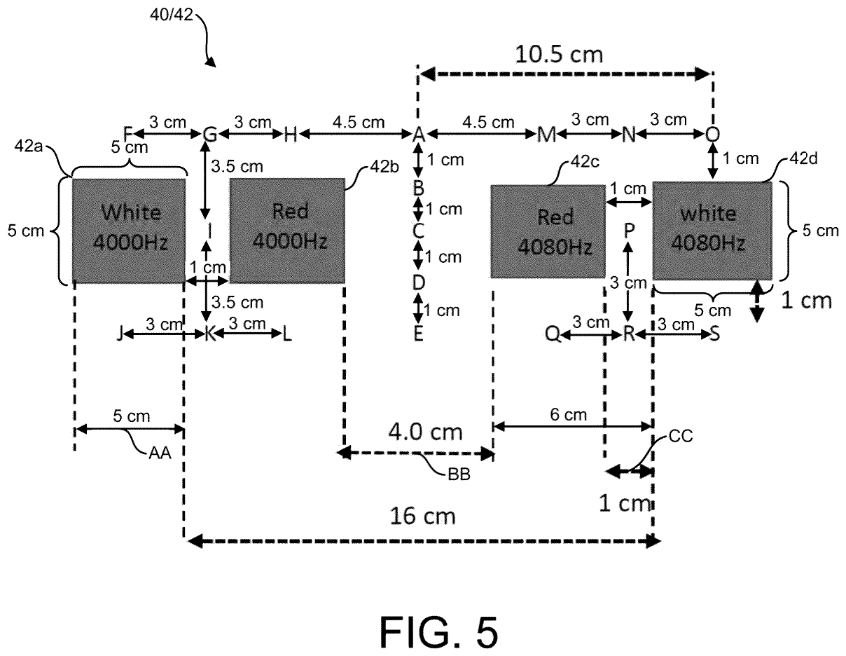

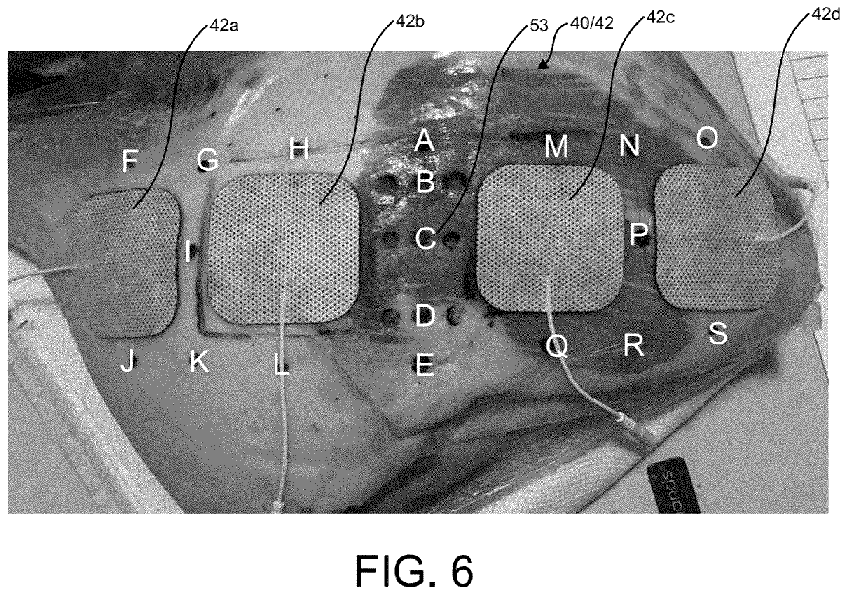

Referring now to FIG. 5, there is shown a lead and electrode configuration 40/42 that was tested using a slab of pork tissue. In FIG. 5, each blue box is a square stimulating electrode 5 cm (or about 2 inches) wide. Each pair of electrodes, with one pair on the left of FIG. 5 and another pair on the right of FIG. 5, was separated by 1 cm (edge to edge). Further dimensions of the test array are shown in FIG. 5. The electrode array on the left of FIG. 5 was separated from the electrode array on the right of FIG. 5 by 4 cm. The white and red electrodes in FIG. 5 refer to cathodes (−) and anodes (+), respectively. The 4000 Hz and 4080 Hz labels in FIG. 5 correspond to the respective carrier frequencies for each electrode. In this example, the difference frequency (or the resulting beat frequency) was 80 Hz. Each letter (A to S) in FIG. 5 corresponds to a location in the slab of pork where a recording electrode was lowered to a depth of 9 cm in 1 cm increments, which 1 cm increments are represented in FIG. 6 for location C in FIG. 5. Note that in the various embodiments electrode polarities may vary. For example, cathodes may not necessarily always be in the red locations and anodes may not necessarily be in the white shown in the accompanying drawings. The locations of electrodes may also vary.

FIG. 6 shows the test setup of FIG. 5 in actual use and deployment on a slab of pork tissue, where all the dimensions set forth in FIG. 5 are employed in the setup of FIG. 6. In FIG. 6, the locations at which recording electrodes were inserted into the slab are represented by locations and corresponding electrode insertion holes A through S as explained above. Electrode pads 42a, 42b, 42c and 42d are also shown in FIG. 6, where each such electrode/electrode pad 42 forms a portion of a lead/electrode 40/42.

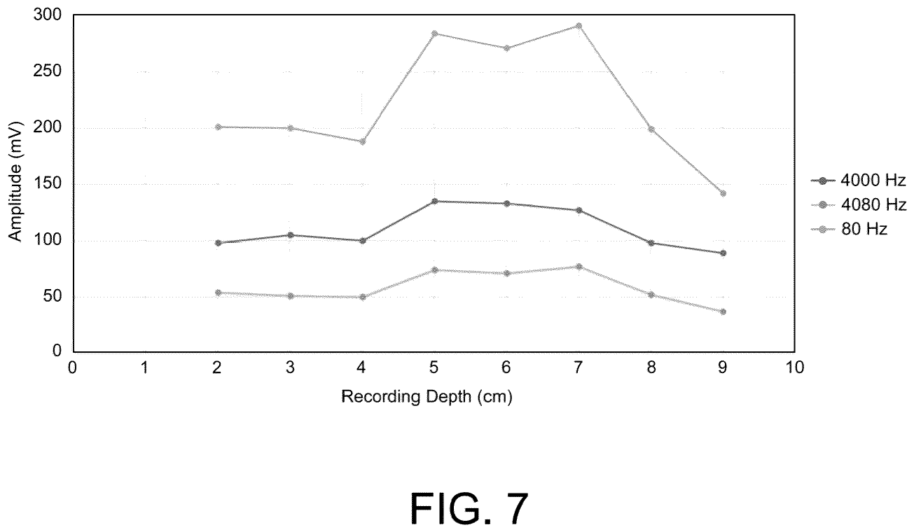

FIG. 7 shows test results obtained using the electrode configurations of FIGS. 5 and 6, where a recording electrode (not shown) was employed at location C of FIGS. 5 and 6 to sense electrical stimulation signals emitted by the electrode configuration of FIG. 5. The stimulation signal amplitudes employed in FIGS. 5, 6 and 7 were 22 mA. In FIG. 7, recording amplitudes in mV are shown as a function of recording electrode depths ranging between 2 cm and 9 cm recorded at location C shown in FIG. 5. FIG. 7 shows that at all tissue depths the combined interferential stimulation beat frequency signal shown in grey produced substantially greater recorded amplitude signals than did the separate 4000 Hz blue stimulation signal or the 4080 Hz yellow stimulation signal.

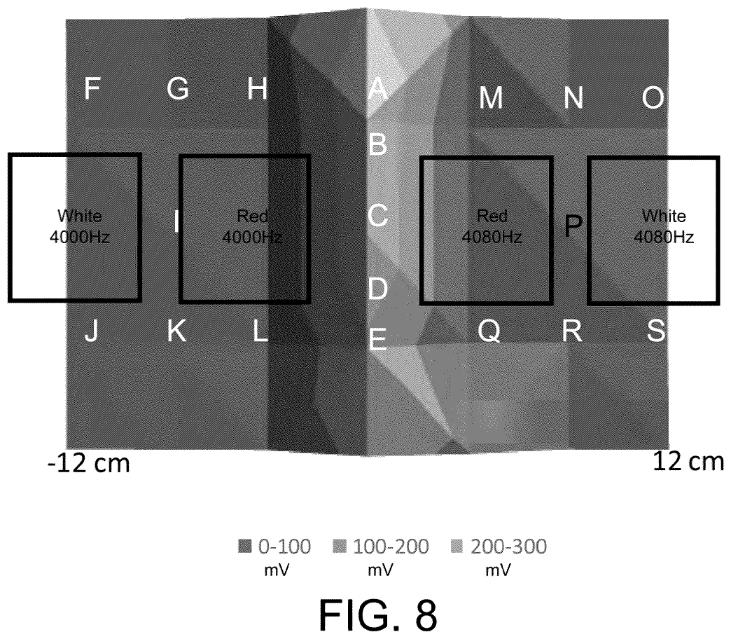

Referring now to FIG. 8, there are shown further test results obtained using the electrode configuration of FIGS. 5 and 6. Beat frequency (80 Hz) amplitude measurements taken at each recording electrode location (locations A to S in FIGS. 5 and 6 shown in white font) were used to generate the contour plot shown in FIG. 8. In FIG. 8, results are shown for a depth of 5 cm only in the tested pork tissue/slab. Blue shading corresponds to beat frequency amplitudes ranging between 0 and 100 mV. Orange shading corresponds to beat frequency amplitudes ranging between 100 and 200 mV. Grey shading corresponds to beat frequency amplitudes ranging between 200 and 300 mV. Note that peak voltages occurred between the left and right pairs of electrodes 40/42 in the middle of FIG. 8, which in one embodiment are located directly above the region of the patient's brain that has experienced a stroke.

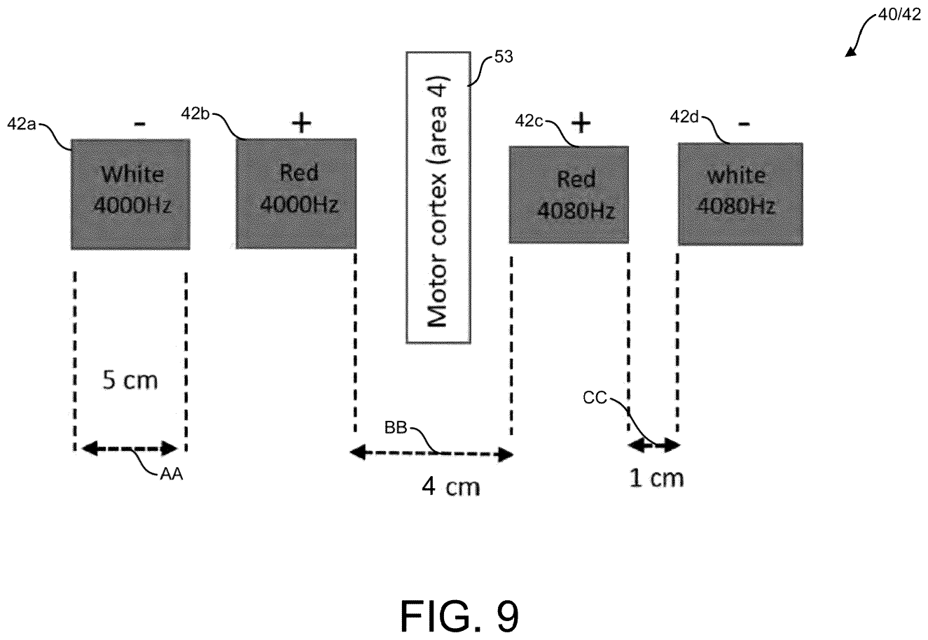

Referring now to FIG. 9, there are illustrated various example of some embodiments of surface electrode configurations 40/42 and 42a-42d. In one embodiment, stimulating electrodes 42a-42d are placed on the surface of the skull, but below the skin. Distance AA corresponds to an electrode diameter/width, which in one embodiment can vary between about 1 mm and about 10 cm. Distance BB corresponds to electrode spacing between electrodes 42a-42d within a pair, which in one embodiment can vary between about 1 mm and about 10 cm. Distance CC corresponds to a distance between electrode pairs, which in one embodiment can vary between about 1 mm and about 10 cm. As described above, electrodes 42a-42d can have various shapes, sizes and configurations, and different numbers of electrodes 42 can be employed (e.g., 2, 4, 6, 8, 10, 12, 14, 16, 18, 20, 22, 24, 26, 28, 30, 32, 34, 36, 38, 40, 42, 44, 46, 48 electrodes and so on). Not all electrodes in an array must be of the same shape, size or configuration. For example, some electrodes may be circular, while others may be square, while still others may be triangular, all within the same array.

In addition, and with reference to FIGS. 1-9, in some embodiments EPG and/or IPG 12 and/or portions thereof may be incorporated into a patch or module that also contains or is operably connected to electrode(s) 42, eliminating the need to connect electrodes 42 to a remotely situated, located, disposed or implanted IPG/EPG 12. Communication between IPG/EPG 12 can also be effected using Bluetooth or other wireless means. Electrical power sources for delivering stimulation signals from electrodes 42 can also be provided by batteries or other power sources such as capacitors located near or on the electrodes or electrode patches 42. In some embodiments, such power sources can be rechargeable.

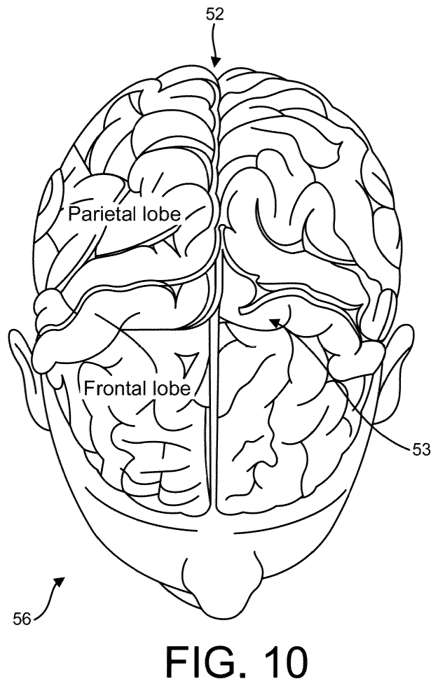

FIG. 10 shows a superior view of the brain of a patient. In FIG. 10, a patient's head 56, cranium and brain 52 are illustrated shown along with the primary motor cortex (also known as Brodmann's area 4) 53. Note that the location of motor cortex area 53 (or Broadman's area 4) is indicated only generally in FIGS. 10, 11 and 12.

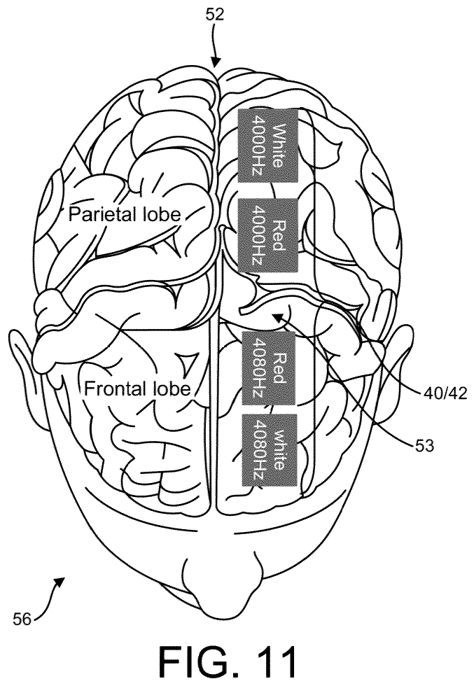

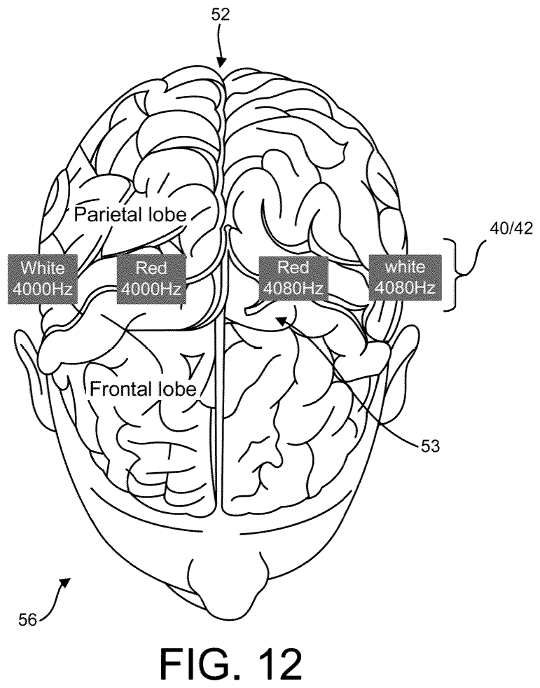

FIGS. 11 and 12 show the cerebral cortex with electrode arrays 40/42 overlain parallel and perpendicular to the motor cortex strip of patient's brain 52, respectively. Electrode arrays 40/42 are preferably situated and/or located on or in the patient's head for optimal or effective interferential electrical stimulation of the portion 50 of patient's brain 52 that has experienced a stroke. In some cases, the electrode array may lie at an angle with respect to the motor strip, as opposed to parallel or perpendicular. The stimulation frequencies and polarity shown in FIGS. 11 and 12 are merely illustrative and not intended to be limiting.

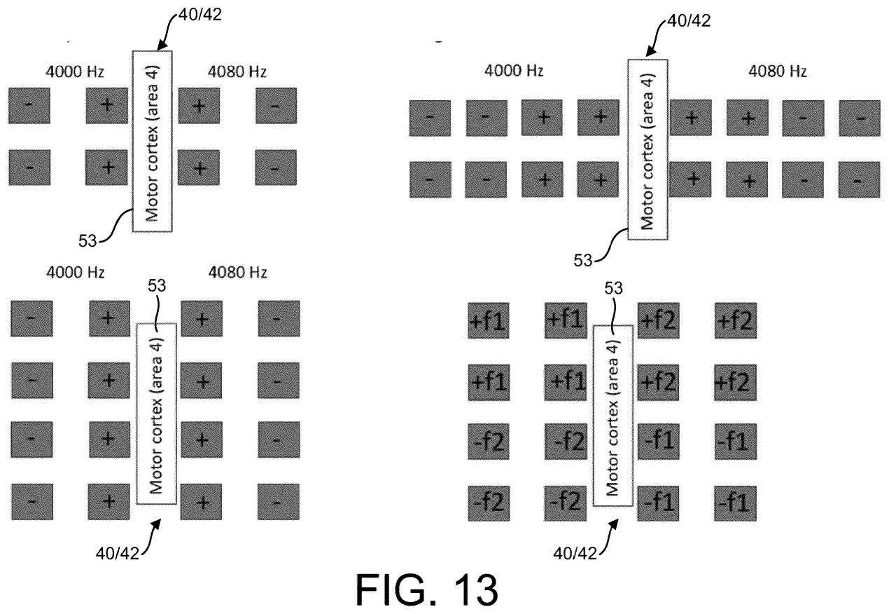

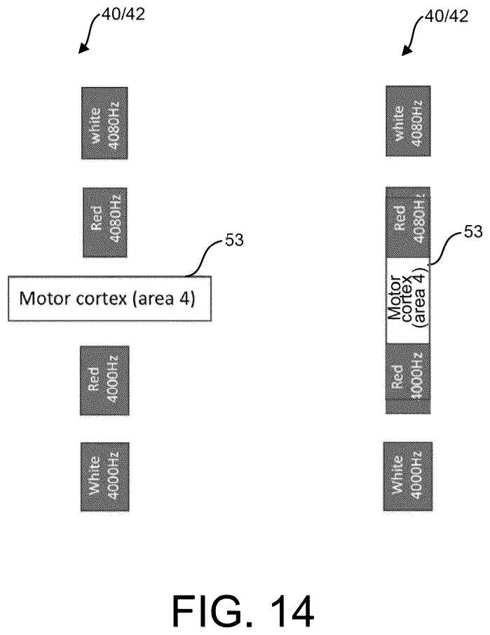

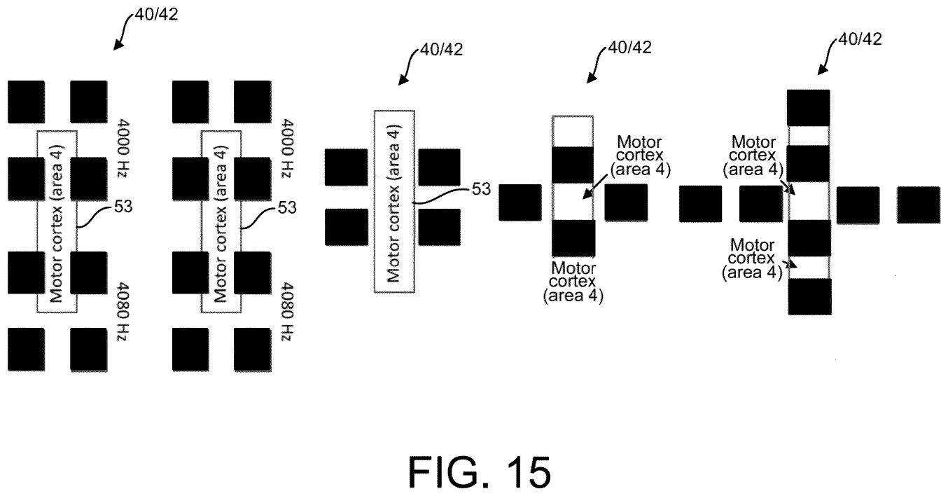

FIGS. 13 through 15 show some illustrative or representative electrode configurations 40/42 that may be employed in some embodiments of the interferential electrical stimulation systems, devices, components and methods described and disclosed herein. FIG. 13 shows electrode configurations 40/42, where electrode arrays are located to either side of the patient's motor cortex or strip 53. FIG. 14 shows further electrode configurations 40/42, where electrode arrays 40/42 are shown perpendicular and parallel to the patient's motor cortex or strip 53. FIG. 15 shows still further electrode configurations 40/42, where electrodes 40/42 overlie the patient's motor cortex or strip 53. The stimulation frequencies (where f1 and f2 are carrier frequencies) shown in FIGS. 13-15 are merely illustrative and not intended to be limiting. Note that in some embodiments more than two carrier frequencies may be employed to deliver electrical stimulation to region 50, such as, by way of non-limiting example, three carrier frequencies, four carrier frequencies, five carrier frequencies, six carrier frequencies, seven carrier frequencies, eight carrier frequencies, nine carrier frequencies, ten carrier frequencies, and so on.

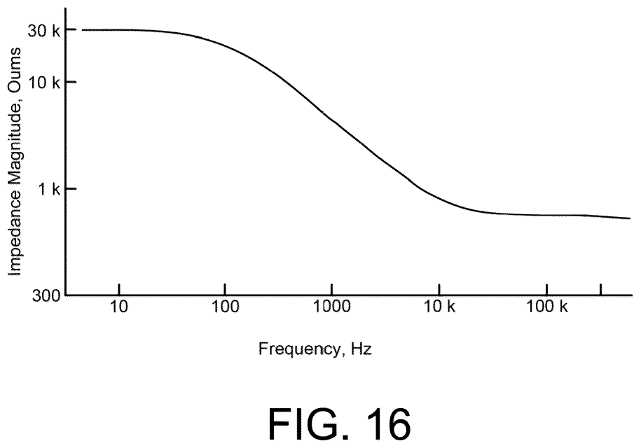

FIG. 16 shows an example of skin impedance (Ohms) as a function of electrical stimulation sinusoidal frequency. As shown in FIG. 16, skin impedance drops as the carrier frequency goes up (adapted from: Webster, Medical Instrumentation: Application and Design, John Wiley & Sons, Inc, New York, 1998). Therefore, higher frequencies pass more readily through the skin (and other high impedance barriers such as bone) than do lower frequencies, which underpins and explains some of the advantages of some embodiments of the interferential electrical stimulation systems, devices, components, and methods described and disclosed herein for delivering therapy and treating stroke in a patient. It is believed that higher stimulation frequencies (e.g., greater than about 1,000 Hz) pass though high impedance barriers more readily than lower stimulation frequencies. However, neurons respond to low stimulation frequencies (e.g., around 100 Hz or less) better than high stimulation frequencies. Hence, it is believed that high frequency carrier frequencies can penetrate high impedance barriers and deliver a low frequency stimulation beat frequency to neurons via the interferential electrical stimulation therapy techniques described and disclosed herein.

See also Haeussinger et al. 2011 [Haeussinger F. B., Heinzel, S., Hahn, T., Schecklmann, and M., Ehlis A-C. et al., 2011, “Simulation of Near-Infrared Light Absorption Considering Individual Head and Prefrontal Cortex Anatomy: Implications for Optical Neuroimaging,” PLOS ONE 6 (10)], which describes some representative scalp, skull and CSF thicknesses in humans. The average thicknesses of scalp, skull and CSF in 24 normal subjects were measured using MRI techniques. Tabulated values in Haeussinger et al. provide an estimate of how deep interferential stimulation therapy must penetrate the scalp, skull and CSF in order to reach the brain and the area of stroke. Accordingly, it has been determined that in some embodiments interferential electrical target stimulation depths to the top surface of the motor cortex or strip 53, as measured from the top surface of a patient's cranial skin to the top surface of the patient's motor cortex or strip 53, may range between about 5 mm and about 30 mm. Note, however, that in some embodiments interferential electrical target stimulation depths go deeper than the top surface of the patient's motor cortex or strip 53, and in some embodiments may extend as deep as about 10 cm (or even deeper).

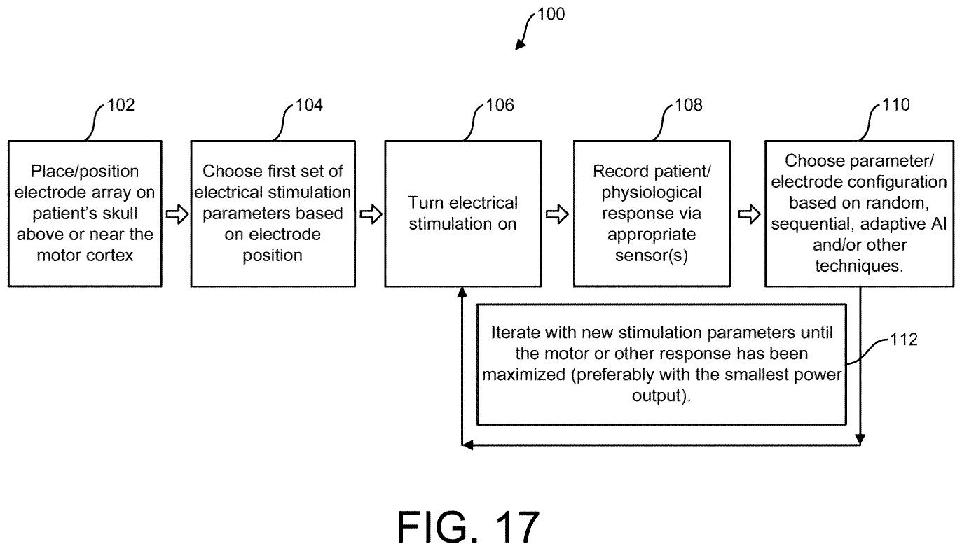

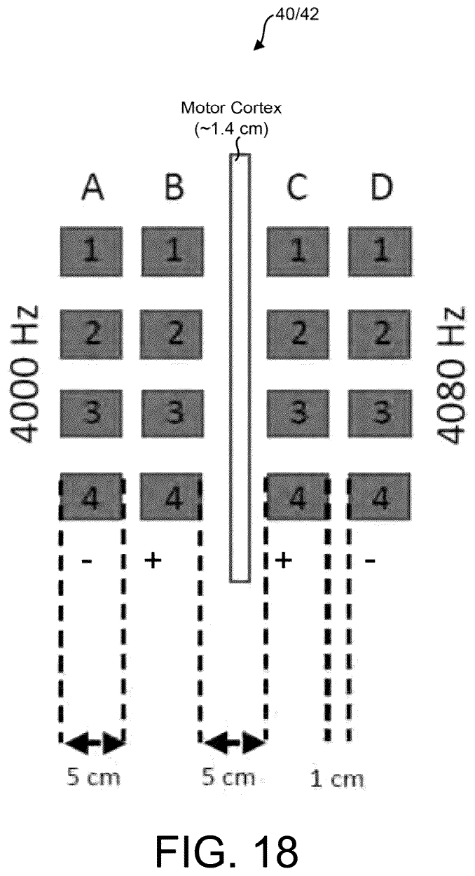

FIGS. 17 and 18 illustrate one embodiment of a method 100 that may be employed to provide interferential electrical stimulation therapy to a patient who has suffered from a stroke.

In FIG. 17, at step 102 a suitable electrode array 40/42 is disposed atop a patient's skull, preferably above the patient's motor cortex or strip 53. At step 104, a first set of stimulation parameters is selected based in part on the electrode array position and configuration that have been selected. At step 106, interferential electrical stimulation is initiated. The patient's response to the interferential stimulation is then recorded, noted or observed using external motion or sound sensors, or by recording the patient's responses provided to a trigger or other button or touch device at step 108. At step 110, still further interferential electrical stimulation parameters and/or electrode configurations are tested in the patient using, for example, closed loop iterative or adaptive techniques, AI techniques, sequential stimulation techniques, adaptive AI techniques, iterative Laplacian convergence techniques, neutral network techniques, and/or other suitable techniques. At step 112, updated or new interferential electrical stimulation parameters and/or electrode configurations (e.g., different electrodes, different pairs of electrodes, selected from among the electrodes contained in the electrode array, or in a new electrode array). Thus, method 100 can be iterative in nature, and permit an optimum set of interferential electrical stimulation and electrode configuration parameters to be converged upon, using, by way of example, iterative Laplacian or other AI techniques.

Continuing to refer to FIG. 17, and of non-limiting example, some of the interferential electrical stimulation parameters that may be varied and/or tested include the following: active or passive electrodes, electrode polarity, electrode location, carrier frequency, number of carrier frequencies, carrier frequency amplitude, difference frequency (or beat frequency), burst stimulation, continuous stimulation, periodic stimulation, and using brief pulse trains (e.g., about 50 ms to about 500 ms in length) to elicit a motor or verbal response or to deliver stimulation therapy. Continuous trains of stimulation may cause tetanic contractions in some patients, but brief pulses (or pulse trains) of stimulation may also elicit a brief motor contraction that can be measured using motion detection techniques. Interferential electrical stimulation may also be delivered in brief epochs (for example, <1 second, but not limited to) to elicit a brief motor response. In still other embodiments, stimulation pulses may range between about 10 Hz and about 100 Hz, with pulse widths ranging between about 10 microseconds and 1000 microseconds. To optimize stimulation locations, in some embodiments pulses as low at 0.5 Hz may be employed. For purposes of delivering stimulation therapy, higher frequencies are more likely to be employed.

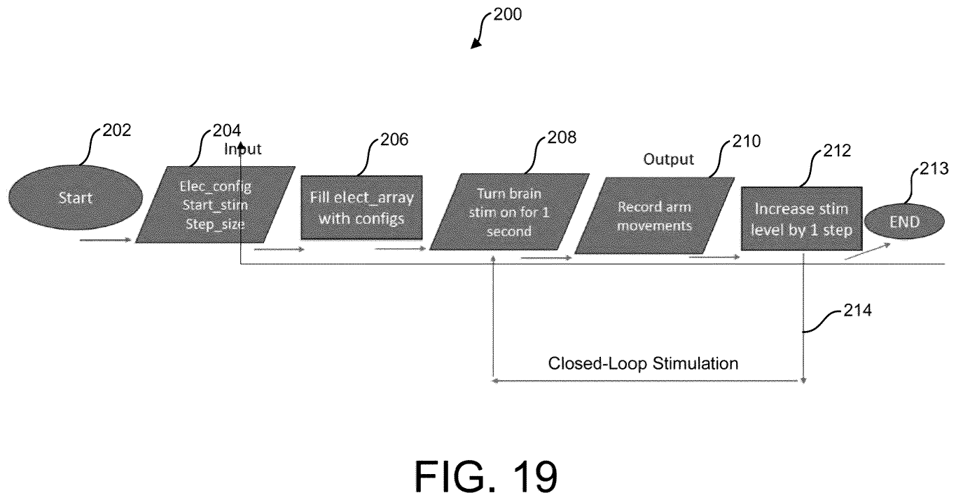

FIG. 18 shows another embodiment of an electrode array 40/42 that may be employed to deliver electrical stimulation to region 50 of a patient. Here, two rows of electrodes 42 are disposed to either side of Broadman's area 4 53. Representative dimensions of electrodes (5 cm per side) and electrode spacing (1 cm) are shown in FIG. 19. Other numbers of electrodes, numbers of rows of electrodes, electrode dimensions, and electrode spacing are contemplated.

Referring again to FIG. 18, and in one some embodiments employing the electrode configuration illustrated in FIG. 18, the 4000 Hz and 4080 Hz carrier frequencies can be e varied between about 1000 Hz and about 50 kHz, and the beat or difference frequency can be varied between about 1 Hz and about 500 Hz. In some electrode configurations, electrodes A1 through A4 of FIG. 18 and electrodes D1 through D4 of FIG. 18 are negative or cathodes (−), whereas electrodes B1 and C1 through B4 and C4 in FIG. 18 are positive anodes (+). Other anode and cathode configurations are also contemplated. In one embodiment, the motor response to various electrode configurations/combinations can be maximized or optimized to achieve the largest motor, muscle twitch, or verbal response. Stimulation parameters can be varied sequentially, randomly, adaptively or using adaptive artificial intelligence techniques (e.g., Laplacian convergence).

Note that the stimulation frequencies shown in FIG. 18 are merely illustrative, and are not intended to be limiting. In addition, the electrical stimulation therapies described and disclosed herein may be provided as open-loop electrical stimulation or as closed loop electrical stimulation to the patient.

FIG. 19 shows another embodiment of a method 200 for providing interferential electrical stimulation therapy to a patient who has suffered from a stroke. One embodiment of pseudo-code corresponding to steps 202 through 213 is set forth below to further illustrate and describe the method 200 illustrated in FIG. 19.

### Example Computer Pseudo-Code According to One Embodiment of a Method of Delivering Interferential Electrical Stimulation to a Patient's Brain to Treat Stroke

#include <stdio. [symbol] >
#include <stdlib. [symbol] >
#include <time. [symbol] >
int main ( ) [symbol]
int h = 10, i = 4, j = 4, k, x, y, z, f = 0;

| | | |
|---|---|---|
| int max_stim_ampl = 0; | /* | Maximum stimulation |

amplitude in mA */

| | | |
|---|---|---|
| int elec_config = 0; | /* | One of 9 electrode |

configurations */

| int start_stim = 0; | | /* The starting |
|---|---|---|
| stimulation amplitude in mA | */ | |
| int step_size = 0; | /* | The size of the step |

(amount) to increase the stimulation amplitude in mA [symbol] /

| int stim_elec_arry[h][i][j]; | /* | The array containing the |
|---|---|---|
| stimulation electrode locations | */ | |
| int arm_mov[10][10] =(0); | /* | The array containing the arm |

| | |
|---|---|
| movement measurement | */ |

```
      srand(time(NULL));
      for (x = 0; x < h; x++) [symbol]
              for (y = 0; y < i; y++) [symbol]
                  for (z = 0; z < j; z++) [symbol]
                    stim_elec_arry[x][y][z] = 0;
                  }
              }
      }
```

| | |
|---|---|
| /* | Enter parameters from |

| | |
|---|---|
| keyboard. Can also read these in from a file | */ |

```
    printf(“Enter electrode configuration (0 to 8): ”);
    scanf(“%d”, &elec_config);
    printf(“you entered %d as the first electrode configuration”,elec_config);
    printf(“\n”);
    printf(“Enter a starting stimulation amplitude (>0): ”);
    scanf(“ [symbol] d”, &start_stim);
    printf(“you entered %d as a starting stimulation amplitude”,start_stim);
    printf(“\n”);
    printf(“Enter the step_size (>0): ”);
    scanf(“%d”, &step_size);
    printf(“you entered %d as an amplitude step size”,step_size);
    printf(“\n”);
    printf(“Enter the max stimulation amplitude (mA) (>0): ”);
    scanf(“%d”, &max_stim_ampl);
    printf(“you entered %d as a maximum stimulation amplitude”,max_stim_ampl);
    printf(“\n”);
/* assign −start_stim and +start_stim to the first array of electrode
locations */
            printf(“Use %d as the first electrode configuration”,elec_config);
            printf(“\n”);
```

| | |
|---|---|
| /* | Configuration 0 |

```
  0 0 0 0
  − + + −
  − + + −
  0 0 0 0
```

*/

```
    if (elec_config==0) {
            stim_elec_arry[0][1][0] = −start_stim;
            stim_elec_arry[0][2][0] = −start_stim;
            stim_elec_arry[0][1][3] = −start_stim;
            stim_elec_arry[0][2][3] = −start_stim;
            stim_elec_arry[0][1][1] = +start_stim;
            stim_elec_arry[0][2][1] = +start_stim;
            stim_elec_arry[0][1][2] = +start_stim;
            stim_elec_arry[0][2][2] = +start_stim;
      }
/* assign −start_stim and +start_stim to the second array of electrode
locations */
```

| | |
|---|---|
| /* | Configuration 1 |

```
  − + + −
  − + + −
  0 0 0 0
  0 0 0 0
```

*/

```
    if (elec_config==1) [symbol]
            stim_elec_arry[1][0][0] = −start_stim;
            stim_elec_arry[1][1][0] = −start_stim;
            stim_elec_arry[1][0][3] = −start_stim;
            stim_elec_arry[1][1][3] = −start_stim;
            stim_elec_arry[1][0][1] = +start_stim;
            stim_elec_arry[1][1][1] = +start_stim;
            stim_elec_arry[1][0][2] = +start_stim;
            stim_elec_arry[1][1][2] = +start_stim;
```

| | |
|---|---|
| } | /* add at up to 10 electrode |

configurations [symbol] */

```
  /* , */
  /* , */
  /* , */
```

/* assign −start_stim and +start_stim to the last (9th) array of electrode
locations */

| | |
|---|---|
| /* | Configuration [symbol] |

```
  − + + −
  − + + −
  − + + −
```

*/

```
    if (elec_config==8) {
            stim_elec_arry[8][0][0] = −start_stim;
            stim_elec_arry[8][1][0] = −start_stim;
            stim_elec_arry[8][2][0] = −start_stim;
            stim_elec_arry[8][3][0] = −start_stim;
            stim_elec_arry[8][0][3] = −start_stim;
            stim_elec_arry[8][1][3] = −start_stim;
            stim_elec_arry[8][2][3] = −start_stim;
            stim_elec_arry[8][3][3] = −start_stim;
            stim_elec_arry[8][0][1] = +start_stim;
            stim_elec_arry[8][1][1] = +start_stim;
            stim_elec_arry[8][2][1] = +start_stim;
            stim_elec_arry[8][3][1] = +start_stim;
            stim_elec_arry[8][0][2] = +start_stim;
            stim_elec_arry[8][1][2] = +start_stim;
            stim_elec_arry[8][2][2] = +start_stim;
            stim_elec_arry[8][3][2] = +start_stim;
      }
```

| | |
|---|---|
| for (z = 0; z < max_stim_ampl; z++) { | /* Begin |

```
For-loop to step through stim amplitudes */
    for (int j = 0; j < 4; j++) {
        for (int k = 0; k < 4; k++) {
              int stim = stim_elec_arry[elec_config][j][k];
              if (abs(stim) <= start_stim [symbol]  f==0) {
```

| | |
|---|---|
| printf(“%d ”, stim;) | /* Mimic Turning |

```
on stimulation electrode array */
              }
            if (stim_elec_arry[elec_config][j][k] > 0) {
                  stim_elec_arry[elec_config][j][k] =
stim_elec_arry[elec_config][j][k] + step_size;
            }
            if (stim_elec_arry[elec_config][j][k] > 0) [symbol]
                  stim_elec_arry[elec_config][j][k] =
stim_elec_arry[elec_config][j][k] − step_size;
            }
        }
        printf(“\n”);
    }
    f=1;
```

/* Assign a random integer between 1 and 10 as

```
mock arm movement data  */
      arm_mov[elec_config][z] = rand( ) % 10;
      printf(“%d”, arm_mov[elec_config][z]);
      printf(“\n”);
```

| | |
|---|---|
| | /* Look for electrode/stimulation configuration |
| with the maximum arm movement | */ |
| | /* This may dictate the optimal therapeutic |

stimulation parameters */

/* print out the updated

```
stimulator array */
    for (int i = 0; i < 4; i++) [symbol]
          for (int j = 0; j < 4; j++) {
                printf(“%d ”, stim_elec_arry[elec_config][i][j]);
          }
          printf(“\n”);
    }
```

| | | |
|---|---|---|
| } | /* | End of For-loop to step |

through stim amplitudes */
return 0;
} [symbol]
indicates data missing or illegible when filed

Thus, and according to some embodiments, there are provided methods of electrically stimulating a portion of a patient's brain to treat stroke. The methods can comprise positioning a plurality of electrodes on a patient's skull; delivering first interferential electrical stimulation signals that combine to form at least one beat frequency associated therewith through at least a first pair or more of the plurality of electrodes to a region of the patient's brain that has been identified as having been affected or likely to have been affected by a stroke; at least one of monitoring, measuring, sensing and recording at least one of verbal responses of the patient and motor movements of one or more limbs of the patient in response to delivery of the first interferential electrical stimulation signals through the at least first pair or more of the plurality of electrodes to the region of the patient's brain that has been identified as having been affected or likely to have been affected by the stroke; selecting, from among the plurality of electrodes, a second pair or more of electrodes different from the first pair or more of electrodes through which to deliver second interferential electrical stimulation signals to the region of the patient's brain that has been identified as having been affected or likely to have been affected by the stroke; at least one of monitoring, sensing, measuring, and recording verbal responses of the patient or motor movements of the limbs of the patient in response to delivery of the second interferential electrical stimulation signals through the at least second pair or more of the plurality of electrodes to the region of the patient's brain that has been identified as having been affected or likely to have been affected by the stroke; subsequently and sequentially repeating delivery of interferential stimulation signals to the region of the patient's brain that has been identified as having been affected or likely to have been affected by the stroke using different pairs or more of electrodes, each pair or more of electrodes being different from a preceding pair or more of electrodes; for each pair or more of electrodes through which interferential electrical stimulation signals are delivered to the region of the patient's brain that has been identified as having been affected or likely to have been affected by the stroke, at least one of monitoring, sensing, measuring and recording verbal responses of the patient and motor movements of the patient's one or more limbs; determining, from among the monitored or measured limb movements of the patient corresponding to interferential electrical stimulation signals delivered through the different pairs or more of electrodes, one or more pairs of electrodes that provide optimum verbal responses of the patient or optimum limb movements in the patient, and selecting the one or more pairs of electrodes that provide at least one of optimum verbal response of the patient and optimum limb movements in the patient for chronic or episodic interferential electrical stimulation of the region of the patient's brain that has been identified as having been affected or likely to have been affected by the stroke, thereby to treat stroke in the patient.

Such methods may further comprises one or more of: (a) wherein the interferential electrical stimulation signals are delivered simultaneously through the at least first pair or more of the plurality of electrodes; (b) wherein at least two different interferential electrical stimulation signals are delivered through at least two pairs or more of the plurality of electrodes; (c) further comprising providing the interferential electrical stimulation signals with a carrier frequency ranging between about 1 kHz and about 50 kHz; (d) further comprising providing the interferential electrical stimulation signals with a beat frequency ranging between about 1 Hz and about 300 Hz; (e) further comprising providing the interferential electrical stimulation signals with an amplitude ranging between about 0.5 mA and about 50 mA; (f) further comprising providing the interferential electrical stimulation signals with an on duty cycle selected from the group consisting of about 1 second, about 2 seconds, about 5 seconds, about 10 seconds, about 20 seconds, about 30 seconds, and about 1 minutes; (g) further comprising providing the interferential electrical stimulation signals with an off duty cycle selected from the group consisting of about 1 second, about 2 seconds, about 5 seconds, about 10 seconds, about 20 seconds, about 30 seconds, and about 1 minute; (h) wherein the region of the patient's brain that has been identified as having been affected or likely to have been affected by the stroke is at or near the motor strip of the cortex of the patient's brain (Brodman's area 4); (i) wherein the region of the patient's brain that has been identified as having been affected or likely to have been affected by the stroke is between about 0.5 centimeters and about 10 centimeters beneath the patient's skull; (j) wherein the plurality of electrodes are positioned subcutaneously between the patient's skin and skull; (k) wherein the plurality of electrodes are positioned transdermally atop the patient's skin and skull; (l) wherein the plurality of electrodes are positioned epidurally or subdurally as regards the patient's skull and brain; (m) wherein the plurality of electrodes comprise between 2 electrodes and 64 electrodes; (n) wherein the plurality of electrodes are mounted on a substrate or patch configured for placement on or in the patient's skull; (o) wherein the steps of delivering, monitoring, selecting, and repeating are controlled and executed by a stimulation algorithm and method; (p) wherein optimum verbal response of the patient and optimum movements in the patient's limb or limbs are determined on the basis of at least one of a range of motion of the limb or limbs, a type of muscular contraction occurring in the limb or limbs, a direction of motion of the limb or limbs, and a degree, type or volume of the patient's verbal response to electrical stimulation; (q) further comprising delivering chronic or episodic interferential electrical stimulation signals through the one or more pairs of electrodes that have been determined to provide optimum or largest limb movements in the patient, thereby to treat stroke in the patient, for a period of time ranging between about 1 day and about 6 months; (r) further comprising administering physical therapy or occupational therapy to the patient, which may be provided in conjunction with electrical stimulation therapy, partially in conjunction with electrical stimulation therapy, or not at all in conjunction with electrical stimulation therapy.

In other embodiments, there are provided systems for electrically stimulating a portion of a patient's brain to treat stroke. The systems can comprise a plurality of electrodes, a pulse generator, at least one lead configured to operably connect the pulse generator to the plurality of electrodes, and a sensor configured to at least one of monitor, measure, sense and record at least one of verbal responses of the patient and motor movements of one or more limbs of the patient in response to delivery of first interferential electrical stimulation signals through at least a first pair or more of the plurality of electrodes, the plurality of electrodes being configured to be placed on a patient's skull, the pulse generator being configured to deliver the first interferential electrical stimulation signals through the at least one lead to at least the first pair or more of the plurality of electrodes to a region of the patient's brain that has been identified as having been affected or likely to have been affected by a stroke, the sensor means for monitoring, measuring, sensing or recording the at least one of verbal responses of the patient and motor movements of one or more limbs of the patient in response to delivery of first interferential electrical stimulation signals through at least a first pair or more of the plurality of electrodes to the region of the patient's brain that has been identified as having been affected or likely to have been affected by the stroke being operably connected to the system, the system being configured to select from among the plurality of electrodes a second pair or more of electrodes different from the first pair or more of electrodes through which to deliver second interferential electrical stimulation signals to the region of the patient's brain that has been identified as having been affected or likely to have been affected by the stroke, the system and the sensor further being configured to at least one of monitor, sense, measure and record at least one of verbal responses of the patient and motor movements of the limbs of the patient in response to delivery of the second interferential electrical stimulation signals through the at least second pair or more of the plurality of electrodes to the region of the patient's brain that has been identified as having been affected or likely to have been affected by the stroke, the system still further being configured to subsequently and sequentially repeat delivery of interferential stimulation signals to the region of the patient's brain that has been identified as having been affected or likely to have been affected by the stroke using different pairs or more of electrodes, each pair or more of electrodes being different from a preceding pair or more of electrodes, thereby to treat stroke in the patient.

Such systems may further comprise one or more of: (a) wherein one of a user selects the second pair or more of electrodes and instructions loaded into a non-transient memory of the pulse generator are employed to select the second pair or more of electrodes; (b) wherein the system is configured such that for each pair or more of electrodes through which interferential electrical stimulation signals are delivered to the region of the patient's brain that has been identified as having been affected or likely to have been affected by the stroke, at least one of monitoring, measuring, and sensing verbal responses of the patient and motor movements of the patient's one or more limbs is carried out while the interferential electrical stimulation signals are being delivered to the region of the patient's brain; (c) wherein the system is configured such that from among the monitored, sensed, measured and recorded verbal responses of the patient and limb movements of the patient, one or more pairs of electrodes that provide optimum or largest verbal responses of and limb movements in the patient are selected for chronic or episodic interferential electrical stimulation of the region of the patient's brain that has been identified as having been affected or likely to have been affected by the stroke, thereby to treat stroke in the patient; (d) wherein the interferential electrical stimulation signals are delivered simultaneously through the at least first pair or more of the plurality of electrodes; (e) wherein at least two different interferential electrical stimulation signals are delivered through at least two pairs or more of the plurality of electrodes; (f) further comprising providing the interferential electrical stimulation signals with a carrier frequency ranging between about 1 kHz and about 50 KHz; (g) further comprising providing the interferential electrical stimulation signals with a beat frequency ranging between about 1 Hz and about 300 Hz; (h) further comprising providing the interferential electrical stimulation signals with an amplitude ranging between about 0.5 mA and about 50 mA; (i) further comprising providing the interferential electrical stimulation signals with an on duty cycle selected from the group consisting of about 1 second, about 2 seconds, about 5 seconds, about 10 seconds, about 20 seconds, about 30 seconds, and about 1 minutes; (j) further comprising providing the interferential electrical stimulation signals with an off duty cycle selected from the group consisting of about 1 second, about 2 seconds, about 5 seconds, about 10 seconds, about 20 seconds, about 30 seconds, and about 1 minute; (k) wherein the region of the patient's brain that has been identified as having been affected or likely to have been affected by the stroke is at or near the motor strip of the cortex of the patient's brain (Brodman's area 4); (l) wherein the region of the patient's brain that has been identified as having been affected or likely to have been affected by the stroke is between about 2 centimeters and about 10 centimeters beneath the patient's skull; (m) wherein the plurality of electrodes are positioned subcutaneously between the patient's skin and skull; (n) wherein the plurality of electrodes are positioned transdermally atop the patient's skin and skull; (o) wherein the plurality of electrodes are positioned epidurally or subdurally as regards the patient's skull and brain; (p) wherein the plurality of electrodes comprise between 2 electrodes and 64 electrodes; (q) wherein the plurality of electrodes are mounted on a substrate or patch configured for placement on or in the patient's skull; (r) wherein at least one of the steps of delivering, monitoring, sensing, measuring, recording, selecting, and repeating is controlled and executed by a stimulation algorithm and method; (s) wherein optimum verbal response of the patient and optimum movements in the patient's limb or limbs are determined on the basis of at least one of a range of motion of the limb or limbs, a type of muscular contraction occurring in the limb or limbs, a direction of motion of the limb or limbs, and a degree, type or volume of the patient's verbal response to electrical stimulation; (t) further comprising delivering chronic or episodic interferential electrical stimulation signals through the one or more pairs of electrodes that have been determined to provide at least one of optimum verbal response of the patient and optimum limb movements in the patient, thereby to treat stroke in the patient, for a period of time ranging between about 1 day and about 6 months, and (u) further comprising administering physical/occupational therapy, which may be provided in conjunction with electrical stimulation therapy, partially in conjunction with electrical stimulation therapy, or not at all in conjunction with electrical stimulation therapy.

Other embodiments, permutations, combinations and variations of the foregoing are also contemplated. By way of non-limiting example, such embodiments, permutations, combinations and variations of the foregoing can include one or more of:

What have been described above are examples and embodiments of the methods, systems, devices and components described and disclosed herein. It is, of course, not possible to describe every conceivable combination of components or methodologies for purposes of describing the invention, but one of ordinary skill in the art will recognize that many further combinations and permutations of the devices and methods described and disclosed herein are possible. For example, the various embodiments may employ external pulse generators or stimulators, or implantable pulse generators. They may employ permanent or temporary components, devices or systems. They may be implanted endoscopically or using other techniques. Leadless embodiments are contemplated, as are embodiments which are configured to operate in conjunction with external or implanted systems, devices or components such as sensors or medical electrical leads. The systems or devices may be wired or wireless, Charging of batteries in implanted devices may be accomplished inductively or transcutaneously.

Accordingly, the devices and methods described and disclosed herein are intended to embrace all such alterations, modifications and variations that fall within the scope of the appended claims. In the claims, unless otherwise indicated, the article “a” is to refer to “one or more than one.”

The foregoing description and disclosure outline features of several embodiments so that those skilled in the art may better understand the detailed descriptions set forth herein. Those skilled in the art will now understand that many different permutations, combinations and variations of the systems, devices, components, methods, procedures and techniques described and disclosed herein fall within the scope of the various embodiments. Those skilled in the art should appreciate that they may readily use the present disclosure as a basis for designing or modifying other processes and structures for carrying out the same purposes and/or achieving the same advantages of the embodiments introduced herein. Those skilled in the art should also realize that such equivalent constructions do not depart from the spirit and scope of the present disclosure, and that they may make various changes, substitutions and alterations herein without departing from the spirit and scope of the present disclosure.

Finally, after having read and understood the present specification and understood the drawings, those skilled in the art will now understand and appreciate that the various embodiments described herein provide solutions to long-standing problems in the effective treatment of stroke in patients.

1. A method of electrically stimulating a portion of a patient's brain to treat stroke, the method comprising:
positioning a plurality of electrodes on a patient's skull;
delivering first interferential electrical stimulation signals that combine to form at least one beat frequency associated therewith through at least a first pair or more of the plurality of electrodes to or near a region of the patient's brain that has been identified as having been affected or likely to have been affected by a stroke;
at least one of monitoring, measuring, sensing and recording at least one of verbal responses, motor movement responses, and muscle twitch responses of the patient in response to delivery of the first interferential electrical stimulation signals through the at least first pair or more of the plurality of electrodes to the region of the patient's brain that has been identified as having been affected or likely to have been affected by the stroke;
selecting, from among the plurality of electrodes, a second pair or more of electrodes different from the first pair or more of electrodes through which to deliver second interferential electrical stimulation signals to the region of the patient's brain that has been identified as having been affected or likely to have been affected by the stroke;
at least one of monitoring, sensing, measuring, and recording at least one of verbal responses, motor movement responses, and muscle twitch responses of the patient in response to delivery of the second interferential electrical stimulation signals through the at least second pair or more of the plurality of electrodes to the region of the patient's brain that has been identified as having been affected or likely to have been affected by the stroke;
subsequently and sequentially repeating delivery of interferential stimulation signals to the region of the patient's brain that has been identified as having been affected or likely to have been affected by the stroke using different pairs or more of electrodes, each pair or more of electrodes being different from a preceding pair or more of electrodes;
for each pair or more of electrodes through which interferential electrical stimulation signals are delivered to the region of the patient's brain that has been identified as having been affected or likely to have been affected by the stroke, at least one of monitoring, sensing, measuring and recording at least one of verbal responses, motor movement responses, and muscle twitch responses of the patient;
determining, from among the monitored or measured verbal responses, motor movement responses, and muscle twitch responses of the patient corresponding to interferential electrical stimulation signals delivered through the different pairs or more of electrodes, one or more pairs of electrodes that provide optimum verbal responses, motor movement responses, and muscle twitch responses of the patient, and
selecting the one or more pairs of electrodes that provide at least one of optimum verbal responses, motor movement responses, and muscle twitch responses for chronic or episodic interferential electrical stimulation of the region of the patient's brain that has been identified as having been affected or likely to have been affected by the stroke, thereby to treat stroke in the patient.

2. The method of claim 1, wherein the interferential electrical stimulation signals are delivered simultaneously through the at least first pair or more of the plurality of electrodes.

3. The method of claim 1, wherein at least two different interferential electrical stimulation signals are delivered through at least two pairs or more of the plurality of electrodes.

4. The method of claim 1, wherein the interferential electrical stimulation signals are delivered continuously over a predetermined period of time.

5. The method of claim 1, wherein the interferential electrical stimulation signals comprise two signals, three signals, four signals, five signals or six signals.

6. The method of claim 1, wherein the interferential electrical stimulation signals comprise one or more of burst signals, triangular wave signals, sinusoidal wave signals, square wave signals, and ramped signals.

7. The method of claim 1, further comprising providing the interferential electrical stimulation signals with a carrier frequency ranging between about 1 kHz and about 50 KHz.

8. The method of claim 1, further comprising providing the beat frequency associated with the interferential electrical stimulation signals range between about 1 Hz and about 500 Hz.

9. The method of claim 1, further comprising providing the interferential electrical stimulation signals with an amplitude ranging between about 0.5 mA and about 50 mA.

10. The method of claim 1, further comprising providing the interferential electrical stimulation signals with an on duty cycle selected from the group consisting of about 1 second, about 2 seconds, about 5 seconds, about 10 seconds, about 20 seconds, about 30 seconds, and about 1 minutes.

11. The method of claim 5, further comprising providing the interferential electrical stimulation signals with an off duty cycle selected from the group consisting of about 1 second, about 2 seconds, about 5 seconds, about 10 seconds, about 20 seconds, about 30 seconds, and about 1 minute.

12. The method of claim 1, wherein the region of the patient's brain that has been identified as having been affected or likely to have been affected by the stroke is at or near the motor strip of the cortex of the patient's brain.

13. The method of claim 9, wherein the region of the patient's brain that has been affected or likely to have been affected by the stroke is identified using one or more of MRI (magnetic resonance imaging), CT (computed tomography), fMRI (functional MRI), DTI (diffusion tensor imaging) and PET (positron emission tomography) techniques.

14. The method of claim 1, wherein the region of the patient's brain that has been identified as having been affected or likely to have been affected by the stroke is between about 0.5 centimeters and about 10 centimeters beneath the patient's skull.

15. The method of claim 1, wherein the plurality of electrodes are positioned subcutaneously between the patient's skin and skull.

16. The method of claim 1, wherein the plurality of electrodes are positioned to stimulate transdermally atop the patient's skin and skull.

17. The method of claim 1, wherein the plurality of electrodes are positioned epidurally or subdurally as regards the patient's skull and brain.

18. The method of claim 1, wherein the plurality of electrodes comprise between 2 electrodes and 64 electrodes.

19. The method of claim 1, wherein the plurality of electrodes are mounted on a substrate or patch configured for placement on or in the patient's skull.

20. The method of claim 1, wherein the steps of delivering, monitoring, selecting, and repeating are controlled and executed by a stimulation algorithm and method.

21. The method of claim 1, wherein optimum verbal response of the patient, optimum movements in the patient's limb or limbs, or optimum twitches in the patient's limbs or muscles are determined on the basis of at least one of a range of motion of the limb or limbs, a type of muscular contraction or twitch occurring in the limb or limbs, a direction of motion of the limb or limbs, and a degree, type or volume of the patient's verbal response to electrical stimulation.

22. The method of claim 1, further comprising delivering chronic or episodic interferential electrical stimulation signals through the one or more pairs of electrodes that have been determined to provide optimum or desired limb or twitch movements in the patient, thereby to treat stroke in the patient, for a period of time ranging between about 1 day and about 6 months.

23. The method of claim 1, further comprising administering physical or occupational therapy to the patient.

24. A system for electrically stimulating a portion of a patient's brain to treat stroke in accordance with the method of claim 1, the system comprising:
a plurality of electrodes configured to be placed on the patient's skull including at least a first pair of electrodes and a second pair of electrodes,
a pulse generator,
at least one lead configured to operably connect the pulse generator to the plurality of electrodes, and
a sensor configured to monitor, measure, sense and/or record at least one of (a) verbal responses of the patient, (b) motor movements of one or more limbs of the patient, and (c) muscle twitch responses of the patient in response to delivery of said first interferential electrical stimulation signals that combine to form at least one beat frequency associated therewith through at least said first pair or more of the plurality of electrodes,
wherein:
the pulse generator is configured to deliver the first interferential electrical stimulation signals through the at least one lead to at least the first pair or more of the plurality of electrodes to a region of the patient's brain that has been identified as having been affected or likely to have been affected by a stroke,
the sensor for monitoring, measuring, sensing and/or recording the at least one of said verbal responses, motor movement responses, and muscle twitch responses of the patient in response to delivery of first interferential electrical stimulation signals through at least a first pair or more of the plurality of electrodes to the region of the patient's brain that has been identified as having been affected or likely to have been affected by the stroke is operably connected to at least one other element in the system,
the system is configured to select from among the plurality of electrodes said second pair or more of electrodes different from the first pair or more of electrodes through which to deliver second interferential electrical stimulation signals to the region of the patient's brain that has been identified as having been affected or likely to have been affected by the stroke,
the system and the sensor are configured to at least monitor, sense, measure and/or record at least one of (a) verbal responses, (b) motor movement responses, and (c) muscle twitch responses of the patient in response to delivery of the second interferential electrical stimulation signals through the at least second pair or more of the plurality of electrodes to the region of the patient's brain that has been identified as having been affected or likely to have been affected by the stroke, and
the system is configured to subsequently and sequentially repeat delivery of interferential stimulation signals to the region of the patient's brain that has been identified as having been affected or likely to have been affected by the stroke using different pairs or more of electrodes, each pair or more of electrodes being different from a preceding pair or more of electrodes, thereby to treat stroke in the patient.

25. The system of claim 24, wherein said system further includes instructions loaded into a non-transient memory of the pulse generator that are employed to select the second pair or more of electrodes.

26-45. (canceled)
